(19) United States
(12) Patent Application Publication
(10) Pub. No.: US 2026/0290562 A1
(43) Pub. Date: Sep. 24, 2026

(54) SYSTEM FOR THE MANAGEMENT OF MENTAL HEALTH DATA

(71) Applicant: 106bh LLC, Humble, TX (US)

(72) Inventors: Jon Nathaniel Ford, Humble, TX (US); Michael Duhon, Humble, TX (US)

(21) Appl. No.: 19/570,920

(22) Filed: Mar. 18, 2026

Related U.S. Application Data
(60) Provisional application No. 63/774,396, filed on Mar. 19, 2025.

(51) Int. Cl. G16H 40/20; G16H 20/70; G16H 70/20

(52) U.S. Cl. CPC G16H 40/20; G16H 20/70; G16H 70/20

(57) ABSTRACT

A system and method for managing mental health data includes a client data management system comprising a client database configured to store mental health data associated with a client, a treatment planning system operatively coupled to the client data management system and configured to generate a treatment plan data record based on the mental health data and a current insurance policy, and a compliance management system operatively coupled to the treatment planning system. The compliance management system periodically accesses, via a network, external sources of insurance policy information to obtain an accessed insurance policy, compares the treatment plan data record with the accessed insurance policy to detect a discrepancy, and in response to detecting the discrepancy, automatically updates the treatment plan data record in the client database to conform to the accessed insurance policy. The compliance management system stores a compliance record documenting the update and stores the accessed insurance policy as a versioned record associated with the update.

### BACKGROUND

Behavioral health care in the United States involves a complex relationship between managed care organizations (“MCOs”), healthcare providers, and regulatory bodies. MCOs manage health plans and coordinate coverage for enrolled members, while providers deliver direct clinical services to clients. The administrative interface between these entities requires ongoing coordination around treatment authorizations, documentation standards, and compliance with insurance coverage requirements.

Treatment plans in the behavioral health context must satisfy both clinical objectives and insurance coverage criteria. Insurance requirements governing behavioral health services can change over time as insurers update their policies, as state regulatory bodies revise administrative codes, and as federal standards evolve. When these requirements change, existing treatment plans may fall out of compliance, potentially resulting in claim denials, treatment delays, or gaps in care. Historically, healthcare providers have been responsible for manually tracking regulatory and insurance changes and updating treatment plans accordingly, which is time-consuming and prone to error.

Behavioral health assessments, such as the Child and Adolescent Needs and Strengths (“CANS”) assessment and the Adult Needs and Strengths Assessment (“ANSA”), are widely used tools for evaluating client needs and informing treatment planning. These assessments involve structured questions that require clinical judgment to score. Variability in how different assessors interpret assessment questions can introduce subjectivity into the scoring process, which in turn can affect the quality of treatment plans derived from the assessment data.

Accordingly, there is a need for systems and methods that reduce the administrative burden on behavioral health providers, improve the consistency of assessment data, and maintain alignment between treatment plans and evolving insurance and regulatory requirements.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 is a diagrammatic view of an exemplary operating environment of an exemplary system for the management of mental health data;

FIG. 2 is a diagrammatic view of an exemplary computer system used in the system of FIG. 1;

FIG. 3 is a graphical view of an exemplary interface screen for use in the system of FIG. 1 and relating to a behavioral health assessment view;

FIG. 4 is a graphical view of an exemplary interface screen for use in the system of FIG. 1 and relating to an observation history view;

FIG. 5 is a graphical view of an exemplary interface screen for use in the system of FIG. 1 and relating to a lessons view;

FIG. 6 is a graphical view of an exemplary interface screen for use in the system of FIG. 1 and relating to a needs assessment inventory view;

FIG. 7 is a graphical view of an exemplary interface screen for use in the system of FIG. 1 and relating to a goals view;

FIG. 8 is a graphical view of an exemplary interface screen for use in the system of FIG. 1 and relating to a treatment time tracking and visit count view;

FIG. 9 is a graphical view of an exemplary interface screen for use in the system of FIG. 1 and relating to a treatment plan metrics view;

FIG. 10 is a graphical view of an exemplary interface screen for use in the system of FIG. 1 and relating to a patient and case notes overview view;

FIG. 11 is a graphical view of an exemplary interface screen for use in the system of FIG. 1 and relating to a re-assessment view;

FIG. 12 is a graphical view of an exemplary interface screen for use in the system of FIG. 1 and relating to an engagement evaluation and progress view;

FIG. 13 is a graphical view of an exemplary interface screen for use in the system of FIG. 1 and relating to a progress-to-target spider chart view;

FIG. 14 is a graphical view of an exemplary interface screen for use in the system of FIG. 1 and relating to an engagement trends view; and

FIG. 15 is a flowchart of a method for the management of mental health data that may be performed by the system of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Administrative Barriers

In the context of behavioral health care, MCOs and healthcare providers often face numerous administrative challenges that can impact patient care. These barriers may include complex authorization processes, inconsistent data collection methods, and varying standards of care across different providers or regions. The present invention has been developed to address these issues by providing a unified platform that standardizes administrative procedures and facilitates seamless communication between MCOs and providers.

The relationship between MCOs and healthcare providers is critical in the delivery of behavioral health services. MCOs typically manage health plans and coordinate care for enrolled members, while providers deliver direct patient care. This relationship can be complex, involving negotiations of service rates, adherence to specific care guidelines, and management of patient authorizations. The present invention is designed to support and enhance this relationship by providing tools that streamline these interactions and promote more efficient collaboration.

Administrative barriers in behavioral health care management can manifest in various forms, such as delays in treatment approvals, difficulties in sharing patient information securely, and challenges in coordinating care across multiple providers. These barriers can lead to fragmented care, increased costs, and potentially poorer patient outcomes. The present disclosure aims to mitigate these issues by offering features that simplify administrative tasks, enhance data sharing capabilities, and support more coordinated care delivery.

By improving patient data collection methodologies, the present invention seeks to enhance the quality and consistency of information available to both MCOs and providers. This may involve standardized assessment tools, structured data entry processes, and integrated reporting systems. The goal is to ensure that all parties involved in a patient's care have access to comprehensive, up-to-date, and accurate information, facilitating more informed decision-making and personalized care planning.

The present invention also focuses on promoting care standardization across various behavioral health contexts. This may involve incorporating evidence-based practices, standardized treatment protocols, and quality metrics into the software's functionality. By encouraging adherence to established standards of care, the system aims to reduce variability in treatment approaches and improve overall care quality.

In summary, the present invention represents a comprehensive approach to addressing the administrative and clinical challenges in behavioral health case management. By focusing on reducing administrative barriers, enhancing data collection, and promoting standardization, the system aims to support more efficient, effective, and patient-centered care delivery in the behavioral health sector.

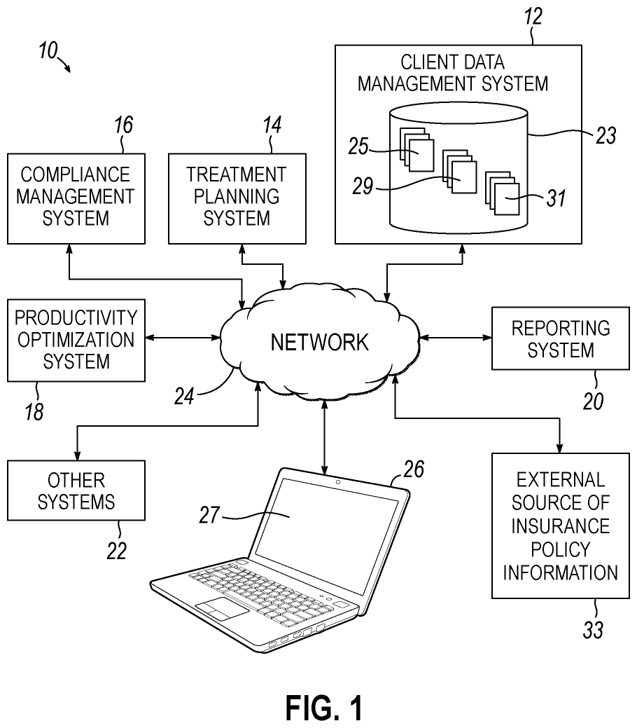

In view of the above issues and inefficiencies, as shown in FIG. 1, a system for the management of mental health data (1), hereinafter referred to as system (1), has been developed to address the administrative barriers in this field. Some versions of system (1) may be embodied or provided by an operating environment (10) utilizing one or more computer systems, as shown in FIG. 1.

### II. Exemplary Computer System

In some versions of operating environment (10), a client data management system (12), a treatment planning system (14), a compliance management system (16), a productivity optimization system (18), a reporting system (20), and an other system (or systems) (22) may send and receive communications between one another directly. Alternatively, in other versions of operating environment (10), client data management system (12), treatment planning system (14), compliance management system (16), productivity optimization system (18), reporting system (20), and other system (22) may communicate with each other through a network (24). Network (24) may include one or more private or public networks (e.g. the Internet) that enable the exchange of data.

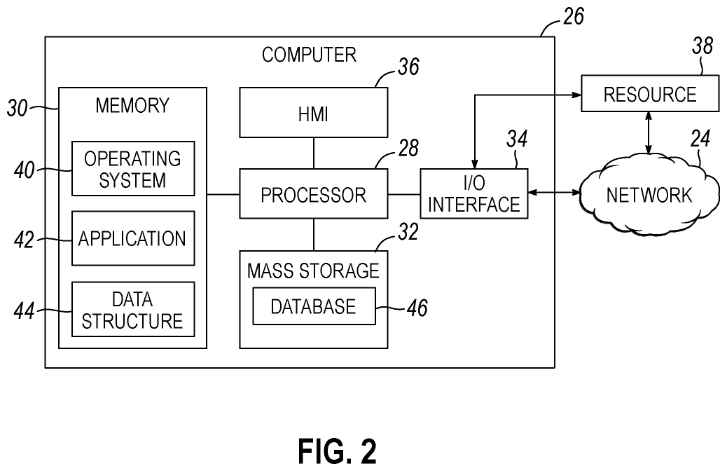

Referring now to FIG. 2, client data management system (12), treatment planning system (14), compliance management system (16), productivity optimization system (18), reporting system (20), and other system (22), and network (24) of operating environment (10) may be implemented on one or more computing devices or systems, such as an exemplary computer system (26). Computer system (26) may include a processor (28), a memory (30), a mass storage memory device (32), an input/output (I/O) interface (34), and a Human Machine Interface (HMI) (36). Computer system (26) may also be operatively coupled to one or more external resources (38) via network (24) or I/O interface (34). External resources may include, but are not limited to, servers, databases, mass storage devices, peripheral devices, cloud-based network services, or any other suitable computer resource that may used by computer system (26).

Processor (28) may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory (30). Memory (30) may include a single memory device or a plurality of memory devices including, but not limited, to read-only memory (ROM), random access memory (RAM), volatile memory, non-volatile memory, static random access memory (SRAM), dynamic random access memory (DRAM), flash memory, cache memory, or any other device capable of storing information. Mass storage memory device (32) may include data storage devices such as a hard drive, optical drive, tape drive, non-volatile solid state device, or any other device capable of storing information.

Processor (28) may operate under the control of an operating system (40) that resides in memory (30). Operating system (40) may manage computer resources so that computer program code embodied as one or more computer software applications, such as an application (42) residing in memory (30), may have instructions executed by processor (28). In an alternative embodiment, processor (28) may execute the application (42) directly, in which case operating system (40) may be omitted. One or more data structures (44) may also reside in memory (30), and may be used by processor (28), operating system (40), or application (42) to store or manipulate data.

I/O interface (34) may provide a machine interface that operatively couples processor (28) to other devices and systems, such as network (24) or external resource (38). Application (42) may thereby work cooperatively with network (24) or external resource (38) by communicating via I/O interface (34) to provide the various features, functions, applications, processes, or modules comprising embodiments of the invention. Application (42) may also have program code that is executed by one or more external resources (38), or otherwise rely on functions or signals provided by other system or network components external to computer system (26). Indeed, given the nearly endless hardware and software configurations possible, persons having ordinary skill in the art will understand that embodiments of the invention may include applications that are located externally to computer system (26), distributed among multiple computers or other external resources (38), or provided by computing resources (hardware and software) that are provided as a service over network (24), such as a cloud computing service.

HMI (36) may be operatively coupled to processor (28) of computer system (26) in a known manner to allow a user to interact directly with computer system (26). HMI (36) may include video or alphanumeric displays, a touch screen, a speaker, and any other suitable audio and visual indicators capable of providing data to the user. HMI (36) may also include input devices and controls such as an alphanumeric keyboard, a pointing device, keypads, pushbuttons, control knobs, microphones, etc., capable of accepting commands or input from the user and transmitting the entered input to processor (28).

A database (46) may reside on mass storage memory device (32), and may be used to collect and organize data used by the various systems and modules described herein. Database (46) may include data and supporting data structures that store and organize the data. In particular, database (46) may be arranged with any database organization or structure including, but not limited to, a relational database, a hierarchical database, a network database, or combinations thereof. A database management system in the form of a computer software application executing as instructions on processor (28) may be used to access the information or data stored in records of database (46) in response to a query, where a query may be dynamically determined and executed by operating system (40), other applications (42), or one or more modules. In an embodiment of system (1), database (46) may comprise a client database (23) comprising mental health data that provides information relating to clients, patients, and treatment planning, and assessment results, as will be discussed in greater detail below.

### III. Client Data Management System

Client data management system (12) of system (1) manages client data in behavioral health settings. Client data management system (12) provides a centralized repository for all client records, adheres to HIPAA compliance standards utilizing encryption and secure servers, implements real-time data syncing allowing multiple authorized users to access and update client information simultaneously, and includes secure data sharing features that enable controlled information exchange with authorized external parties.

As shown in FIG. 1, client data management system (12) includes client database (23). Client database (23) is configured to store and organize a set of mental health data associated with one or more clients, which may include demographic information, medical history, and assessment results. Client database (23) further stores treatment plan data records (25), compliance records (29), and versioned records (31), as described further herein. Client database (23) may be implemented using database (46) residing on mass storage memory device (32), or may be a logically distinct data store within database (46). Client database (23) may be accessed by treatment planning system (14), compliance management system (16), productivity optimization system (18), and reporting system (20) to read and write data as needed to support their respective functions within system (1). While client database (23) is shown within client data management system (12) for exemplary purposes, client database (23) may reside within any portion of system (1) or may be a separate element accessible through network (24).

The client intake process in client data management system (12) begins with functionality to initiate a new client intake in compliance with local and federal standards and rules. In some cases, the client intake process includes prompting the user to acknowledge a non-disclosure agreement before accessing the web-based software platform to ensure confidentiality and legal compliance from the outset.

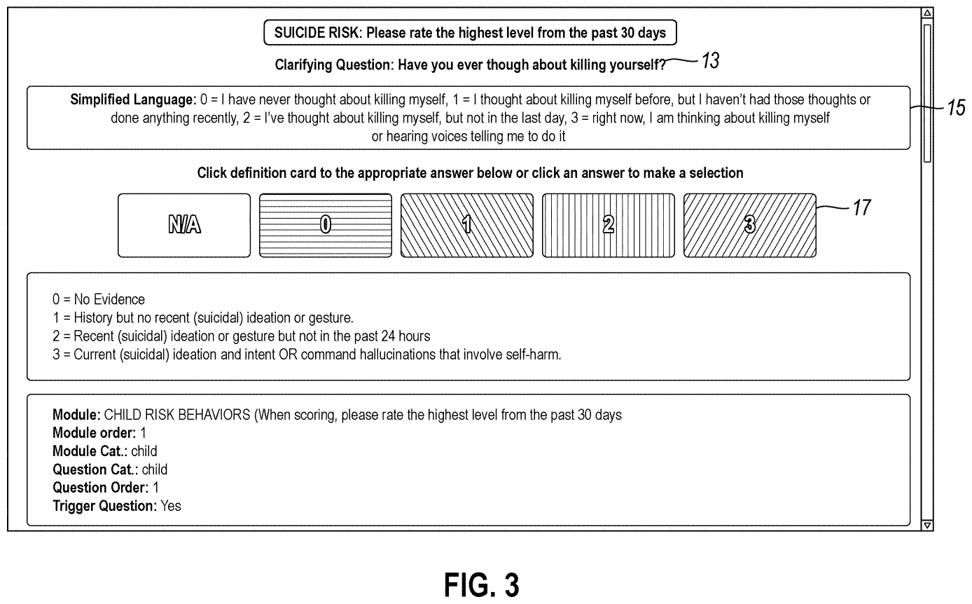

Following initial data entry, client data management system (12) guides users through a behavioral health assessment, which may be a CANS assessment, an ANSA, a Needs Assessment, a Naomi Assessment, or similar, depending on the underlying client, jurisdictional requirements, and rules. As shown in FIG. 3, client data management system (12) presents each assessment question with three concurrent usability enhancements designed to reduce inter-rater subjectivity and improve the consistency of assessment data collected across different assessors and clinical settings.

The first enhancement is clarifying question information, depicted in FIG. 3 within a box (13). Clarifying question information provides additional clinical context for the assessor by explaining the intent behind each assessment question and offering examples of what the question is designed to capture. This information may be displayed as an expandable or hoverable element adjacent to the assessment question text within web interface (27), such that the assessor can access the additional context without navigating away from the current question.

The second enhancement is simplified language, depicted in FIG. 3 within a box (15). The simplified language reformulates each assessment question using plain, accessible terms so that assessors with varying levels of clinical training can more accurately interpret and respond to the question. The simplified language may be displayed below or alongside the standard clinical language of the assessment question.

The third enhancement is a set of selection buttons (17) (FIG. 3), where each possible response may be presented as an individually colored button corresponding to the severity level of the response. For instance, a response indicating no evidence of a need may be displayed in green, a response indicating some history may be displayed in yellow, a response indicating a current need may be displayed in orange, and a response indicating an acute or crisis-level need may be displayed in red. Selection buttons (17) may be arranged in a horizontal or vertical array, with each button labeled with both a numeric value (e.g., 0, 1, 2, 3) and a brief descriptor (e.g., “No Evidence,” “History,” “Current,” “Acute”). When an assessor selects a particular selection button (17), client data management system (12) records both the numeric value and a timestamp in client database (23), enabling subsequent analysis of assessment patterns and inter-rater reliability.

### IV. Treatment Planning System

Treatment planning system (14) of system (1) simplifies and enhances the creation of personalized treatment plans for clients. By integrating client data in real time from client database (23), treatment planning system (14) automates much of the treatment planning process, ensuring that plans are always up to date and reducing the administrative workload for healthcare providers.

Treatment planning system (14) generates and stores a treatment plan data record (25) in client database (23) for each client. Treatment plan data record (25) is a structured data object that includes parameters defining the client's personalized treatment plan, which may include, for example, covered service types, session frequency, session duration limits, required documentation elements, required assessment intervals, billing codes, treatment goals, and assigned providers. Treatment plan data record (25) is generated based at least in part on the set of mental health data stored in client database (23) and on a current insurance policy applicable to the client. Treatment plan data record (25) may be updated by treatment planning system (14) in response to changes in client data, or by compliance management system (16) in response to detected discrepancies between treatment plan data record (25) and an accessed insurance policy, as described further below.

In some cases, treatment planning system (14) may include customizable treatment templates for different behavioral health conditions, ranging from anxiety and depression to substance use disorders and more complex psychiatric issues. These templates serve as starting points that healthcare providers may modify as needed, adding or removing elements to create a personalized plan for each client. Treatment planning system (14) may also feature automated data integration capabilities, seamlessly pulling relevant data from client profiles and other sources within system (1) to ensure that treatment plans are based on the most current and comprehensive client information available.

In some implementations, treatment planning system (14) may incorporate outcome-based planning features that allow for dynamic adjustments to treatment plans based on client progress and measurable goals. Treatment planning system (14) may track client outcomes over time and suggest modifications to the treatment plan as needed, helping healthcare providers make informed, evidence-based decisions about the effectiveness of current treatments. Treatment planning system (14) may also facilitate multidisciplinary collaboration by providing a platform for psychiatrists, psychologists, social workers, and other specialists to contribute their expertise to a client's treatment plan. System (1) may manage access permissions and version control, allowing team members to work together while maintaining the integrity and security of client information.

To support regulatory compliance, treatment planning system (14) in connection with compliance management system (16) may automatically check treatment plan data records (25) against current compliance standards and flag any potential issues for review, reducing the risk of claim denials and regulatory violations while saving time for healthcare providers who would otherwise need to manually verify compliance for each treatment plan.

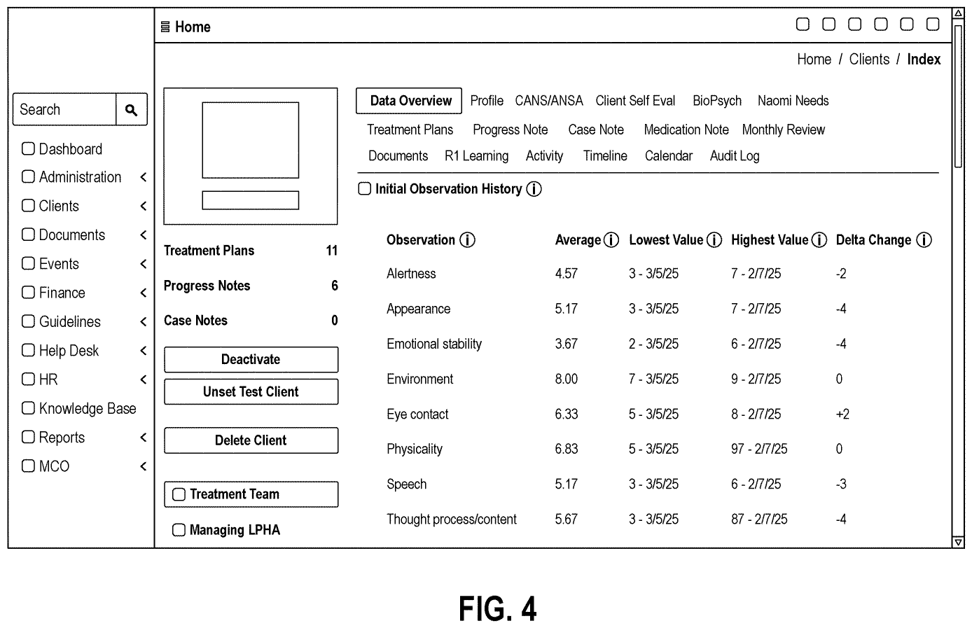

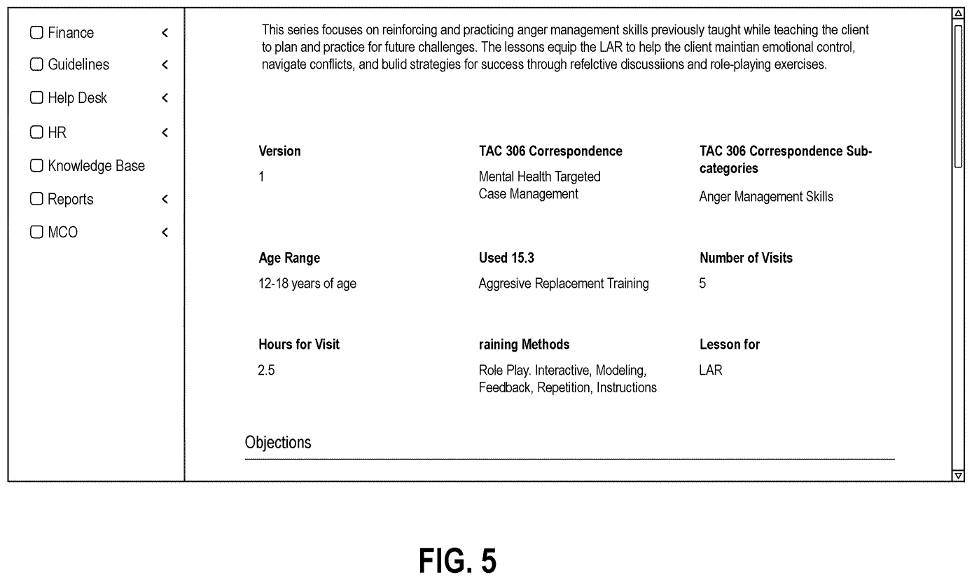

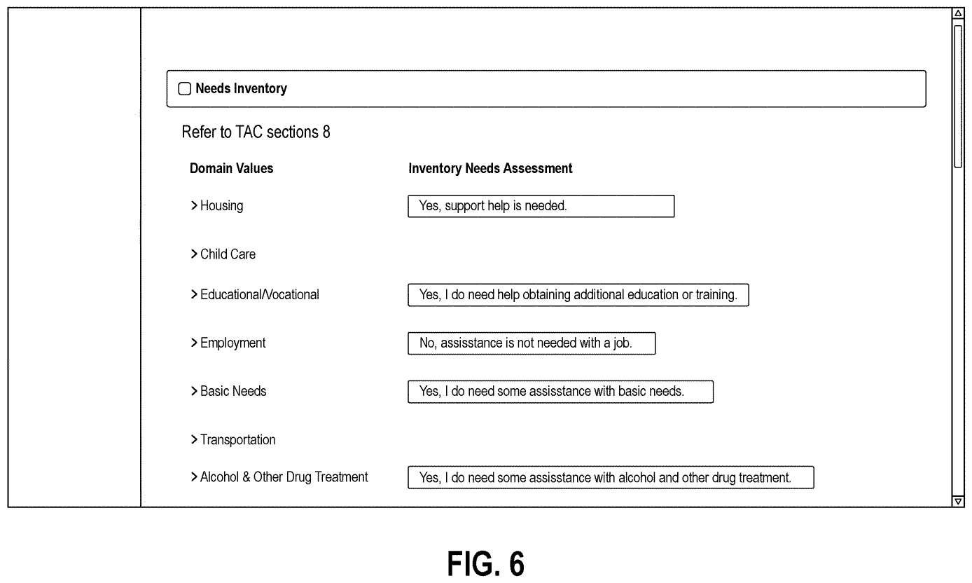

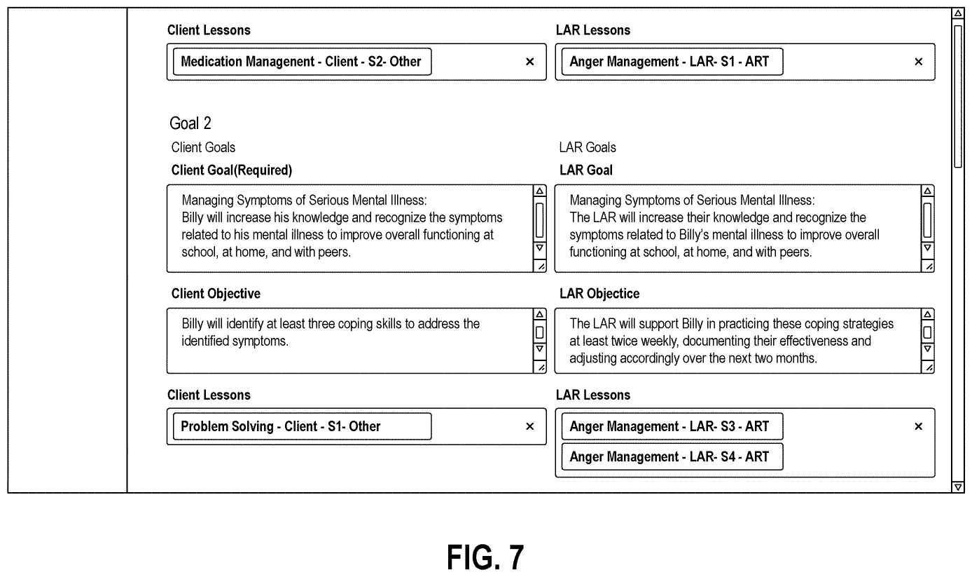

Exemplary web interface (27) screens for treatment planning system (14) are shown in FIGS. 4-7. FIG. 4 illustrates an observation history view displaying a table of clinical observation categories (e.g., abstinence, appearance, emotional stability, eye contact, verbal tracking, speech, thought process/content, and alertness), with columns for average scores, lowest and highest recorded values with corresponding dates, and delta change over time, enabling healthcare providers to track and analyze client progress across multiple clinical dimensions. FIG. 5 illustrates a client/LAR (Legally Authorized Representative) lessons view, displaying treatment lesson details including a version number, applicable Texas Administrative Code (TAC) correspondence and sub-categories, age range, number of visits, hours per visit, training methods (e.g., role play, interactive modeling, feedback, repetition, instructions), and lesson objectives. FIG. 6 illustrates a needs assessment inventory view referencing applicable TAC sections and displaying domain values (e.g., housing, child care, educational/vocational, employment, basic needs, transportation, and alcohol and other drug treatment) as a bar chart, providing a visual representation of client needs across multiple life domains. FIG. 7 illustrates a client goals and LAR goals view, displaying client goals with associated disorder goals, client objectives, and client lessons alongside corresponding LAR goals, LAR objectives, and LAR lessons, supporting a collaborative approach to treatment that engages both the client and their legally authorized representative.

As shown in FIGS. 4-7, web interface (27) of treatment planning system (14) incorporates direct links to relevant sections of the Texas Administrative Code governing rules and regulations. In some implementations, web interface (27) embeds direct hyperlinks to specific sections of governing administrative code that are relevant to a particular treatment plan or treatment plan element. For example, if a treatment plan for a client in the state of Texas includes a particular type of behavioral health service, web interface (27) may display a hyperlink to the relevant section of the Texas Administrative Code that governs the provision of that service. These hyperlinks may be displayed adjacent to the treatment plan element to which they relate, such that a healthcare provider can review the regulatory authority for a treatment plan requirement with a single click, without navigating away from the treatment planning interface of web interface (27). The hyperlinks may be dynamically generated by treatment planning system (14) based on the jurisdiction, service type, and treatment plan content, so that different treatment plans display links to different regulatory provisions as appropriate.

### V. Compliance Management System

Compliance management system (16) of system (1) dynamically adapts treatment plans to align with insurance requirements and regulatory standards. Compliance management system (16) incorporates automated compliance checks that continuously monitor and evaluate treatment protocols against current regulations, accesses up-to-date policy information and requirements through insurance integration, and enables real-time adjustments to treatment plans to ensure that prescribed treatments remain within approved guidelines, minimizing claim rejections and delays.

As shown in FIG. 1, compliance management system (16) communicates via network (24) with one or more external sources of insurance policy information (33). An external source of insurance policy information (33) may be any system, server, website, database, API endpoint, or data feed located external to system (1) that provides information about insurance policies, coverage requirements, regulatory standards, or related guidelines applicable to behavioral health services. By way of example and not limitation, external source of insurance policy information (33) may include an insurance provider website, a state regulatory agency website (e.g., a state department of insurance or state health and human services agency website), a federal regulatory database (e.g., a CMS resource), an API provided by an insurance provider, or an electronic data feed published by an insurance provider or regulatory body. Compliance management system (16) periodically accesses external sources of insurance policy information (33) via network (24) to obtain an accessed insurance policy, which is then used to evaluate and, if necessary, update treatment plan data records (25) stored in client database (23), as described in further detail below.

In some cases, compliance management system (16) may provide direct integration with insurance providers to align treatment plans with coverage requirements, automatically retrieving and interpreting insurance policy details to ensure that treatment plans are tailored to meet specific coverage criteria. In other versions, a user is prompted to confirm insurance regulations are up to date or prompted to upload the latest version. Compliance management system (16) may also feature real-time insurance regulations collection by automatic web crawling of websites relating to insurance information, staying current with changing insurance policies and regulatory standards, automatically updating its internal guidelines, and prompting necessary modifications to existing treatment plans.

Compliance management system (16) implements a closed-loop process for maintaining alignment between treatment plans generated by treatment planning system (14) and evolving insurance requirements. This closed-loop process includes: continuously monitoring one or more external sources of insurance policy information (33) via network (24); detecting changes in insurance requirements that are relevant to existing treatment plans within system (1); automatically adapting affected treatment plans to conform to the changed requirements; and generating compliance documentation reflecting each adaptation.

The continuous monitoring may be performed on a scheduled basis (e.g., daily, weekly) or in response to a trigger event (e.g., a notification from an insurance provider, a user-initiated compliance check). Compliance management system (16) may access external sources of insurance policy information (33) by crawling the web, by querying an API provided by an insurance provider, or by receiving electronic notifications or data feeds from insurance providers or regulatory bodies. When compliance management system (16) detects a change in an insurance requirement (e.g., a change in covered services, a change in required documentation, a change in maximum session limits, or a change in required assessment protocols), compliance management system (16) compares the detected change against the current parameters of each treatment plan data record (25) stored in client database (23) to identify any treatment plans that are affected by the change.

For each affected treatment plan, compliance management system (16) may automatically adapt the treatment plan by modifying one or more of the following exemplary parameters in treatment plan data record (25): covered service types, session frequency, session duration limits, required documentation elements, required assessment intervals, or billing codes. The adaptation may be performed automatically without user intervention, or compliance management system (16) may present a proposed adaptation to a healthcare provider via web interface (27) for review and approval before the adaptation is finalized. Compliance management system (16) may further be configured to automatically update internal compliance guidelines and rules stored in client database (23) in response to detected changes in insurance requirements. For example, if a state regulatory body updates the maximum number of covered visits per treatment episode for a particular behavioral health condition, compliance management system (16) may update an internal rules table that governs treatment plan generation and compliance checking, such that all future treatment plan data records (25) generated by treatment planning system (14) reflect the updated requirement without further manual configuration.

The record of each adaptation generated by compliance management system (16) is stored in client database (23) as a compliance record (29). Each compliance record (29) documents a specific update to a treatment plan data record (25), including the detected discrepancy between treatment plan data record (25) and the accessed insurance policy, the original treatment plan parameters prior to the update, the updated treatment plan parameters following the update, and a timestamp indicating when the update was performed. Compliance records (29) may be associated in client database (23) with the treatment plan data record (25) to which they relate, enabling a healthcare provider, compliance auditor, or other authorized user to review the complete history of compliance-driven modifications to any given treatment plan via web interface (27). Over time, client database (23) may accumulate multiple compliance records (29) for a single treatment plan data record (25), providing a chronological audit trail of all insurance-driven adaptations applied to that treatment plan.

Each accessed insurance policy retrieved by compliance management system (16) from external source of insurance policy information (33) is stored in client database (23) as a versioned record (31). Each versioned record (31) includes the content of the accessed insurance policy, an identifier of the external source of insurance policy information (33) from which it was retrieved, and a retrieval timestamp. When compliance management system (16) updates a treatment plan data record (25) in response to a detected discrepancy, the update is associated in client database (23) with the specific versioned record (31) that prompted the update. Over time, client database (23) accumulates a chronological history of versioned records (31), and each compliance record (29) may reference the corresponding versioned record (31), enabling system (1) to trace any treatment plan modification back to the specific version of the insurance policy that required it. This versioned history may be accessed via web interface (27) to verify that a particular treatment plan was compliant with the insurance policy in effect at a given point in time.

Compliance management system (16) maintains comprehensive documentation of all compliance-related activities, including records of compliance checks, treatment plan adjustments, and communications with insurance providers. System (1) is designed with built-in transparency features to enable thorough auditing by various stakeholders, including attorneys general. System (1) may be configured to maintain audit logs of all user actions, data modifications, and compliance-related decisions, ensuring data integrity throughout the auditing process. System (1) may also incorporate specialized interfaces tailored for use by attorneys general or other authorized auditors, providing streamlined access to relevant compliance data, treatment histories, compliance records (29), versioned records (31), and financial records. These interfaces may include advanced search and filtering capabilities, allowing authorized auditors to efficiently identify and investigate potential compliance issues across multiple healthcare providers or regions.

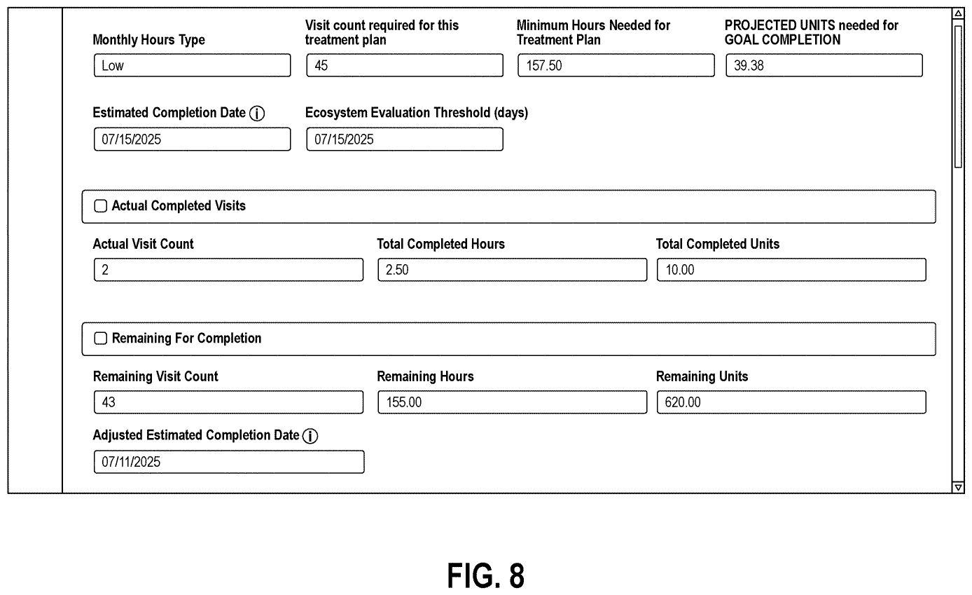

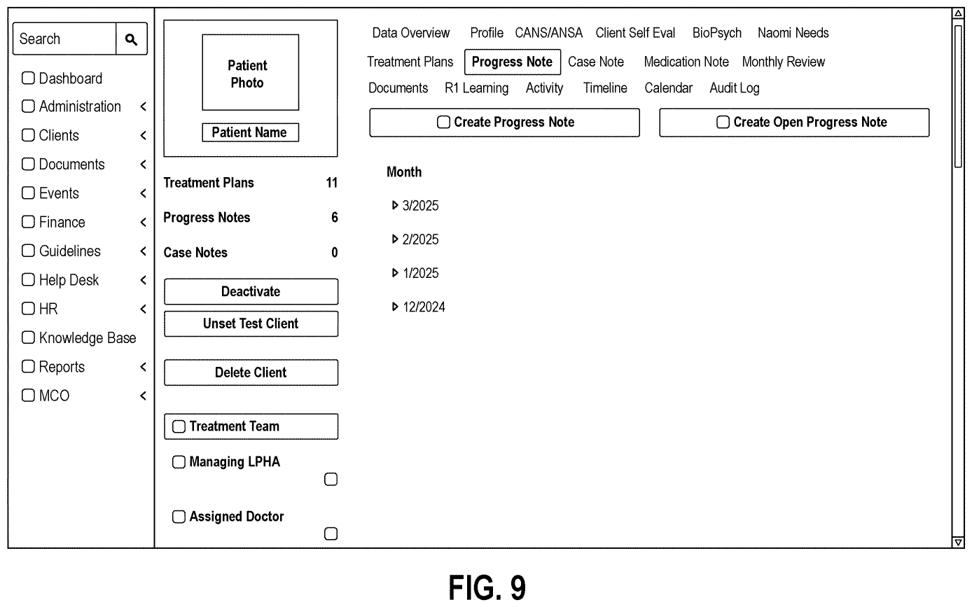

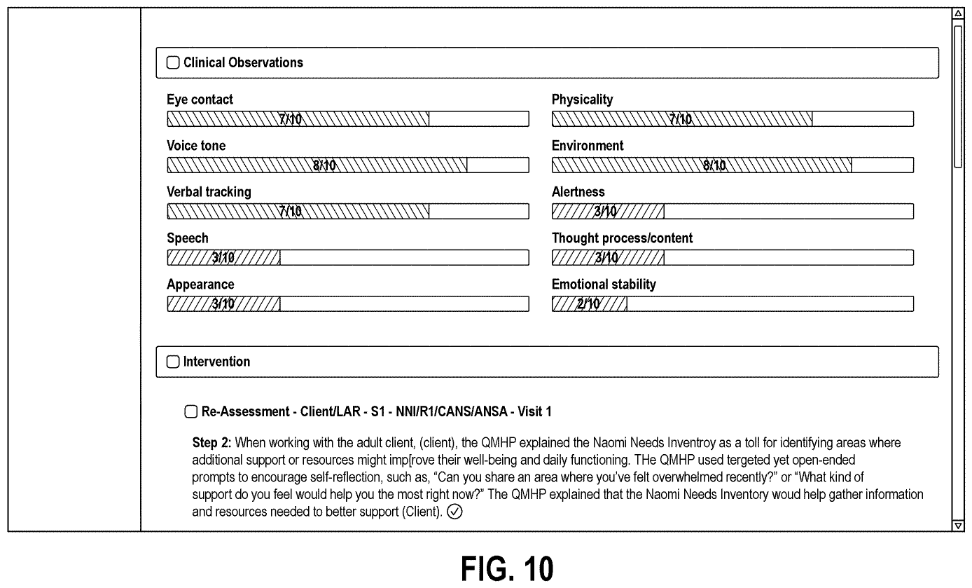

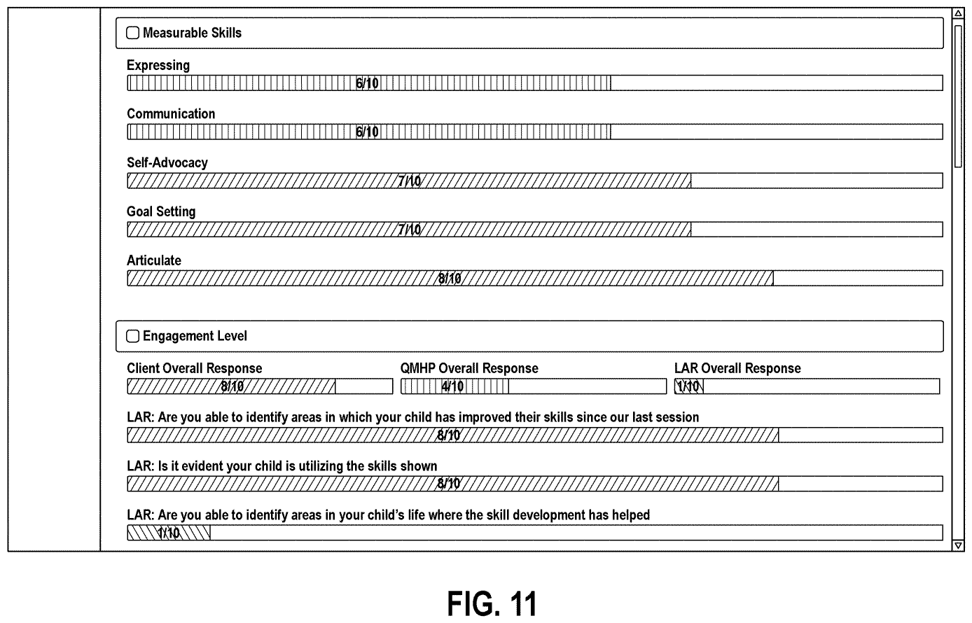

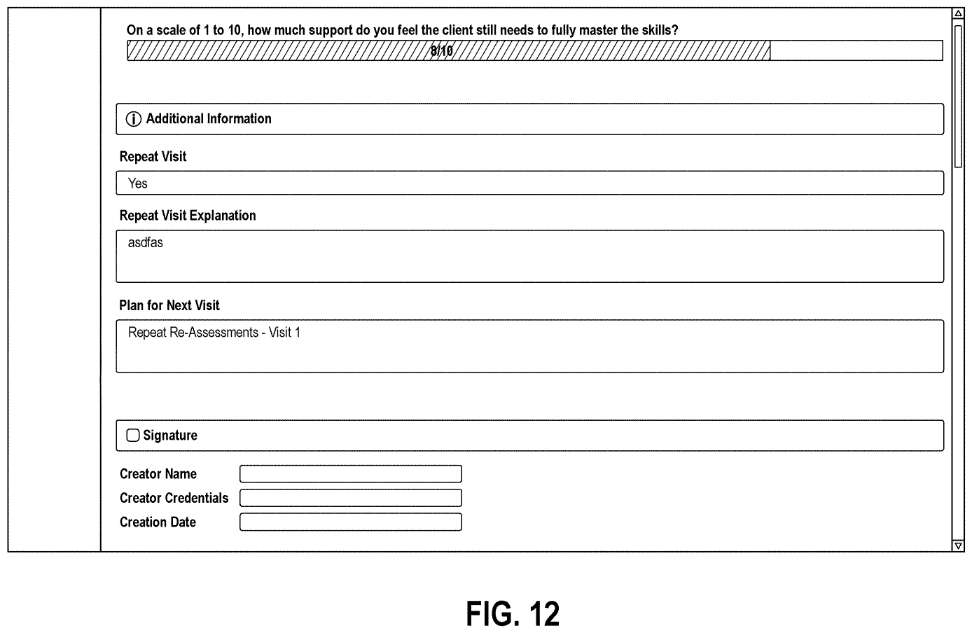

Exemplary web interface (27) screens for compliance management system (16) are illustrated in FIGS. 8-12. FIG. 8 illustrates a treatment plan metrics view displaying visit counts required for a treatment plan, ecosystem evaluation thresholds, estimated completion dates, minimum hours needed for the treatment plan, projected units needed for goal completion, actual visit counts, total completed hours and units, and remaining visits, hours, and units. FIG. 9 illustrates a similar treatment plan metrics view for a different treatment plan configuration, displaying the same categories of metrics with different values, demonstrating the system's ability to track and project treatment plan progress across varying plan types. FIG. 10 illustrates a client overview displaying case notes, treatment plans, progress notes, medication notes, and activity logs organized by month, along with assigned provider information, providing a consolidated view of all client-related data within compliance management system (16). FIG. 11 illustrates a re-assessment view displaying clinical observation metrics (e.g., eye contact, voice tone, verbal tracking, speech, appearance, physicality, environment, alertness, thought process/content, and emotional stability) as a bar chart, alongside a narrative summary of the assessment session describing the client's presentation, engagement, and clinical observations. FIG. 12 illustrates an engagement evaluation and progress view displaying skill metrics (e.g., expressing, communication, self-advocacy, goal setting, and articulation) with corresponding scores, along with client overall response ratings, LAR overall response ratings, and qualitative questions directed to the LAR regarding the client's skill improvement and the impact of skill development on the client's life.

### VI. Productivity Optimization System

Productivity optimization system (18) of system (1) enhances workplace efficiency and streamlines operations in behavioral health case management by incorporating time-saving features that reduce administrative burden, minimize burnout, and foster improved team collaboration.

In some cases, productivity optimization system (18) may include automated scheduling and task management functionality, allowing for the automatic allocation of tasks and scheduling of appointments based on client needs, provider availability, and treatment plans. Productivity optimization system (18) may also incorporate real-time data synchronization capabilities, ensuring that all team members have access to the most current client information, treatment plan data records (25), and task statuses across various devices and platforms. For instance, when a provider updates a client's treatment plan, this information may be instantly available to all authorized team members, facilitating seamless collaboration and continuity of care. In some implementations, productivity optimization system (18) may include performance analytics tools that provide insights into individual and team productivity patterns, workload distribution, and efficiency metrics, enabling managers to make informed decisions about resource allocation and process improvements.

Productivity optimization system (18) may include functionality to automatically communicate with members of the client's support network, such as family members or other trusted individuals, by sending automated emails or text messages requesting these individuals to complete online evaluations regarding the client's progress and well-being. Productivity optimization system (18) may also include an automated post-treatment follow-up module that transmits communications (e.g., emails or text messages) via network (24) to clients who have exited a treatment program, whether by successful completion or early discontinuation. The post-treatment follow-up module is configured to transmit these communications at predetermined intervals (e.g., 30 days, 60 days, 90 days, 6 months after exit) and to collect information about the client's current status, including whether the client is maintaining progress, experiencing a recurrence of symptoms, or in need of additional intervention. If the collected information indicates that a client may benefit from further care, the post-treatment follow-up module may generate a notification to the client's former treatment provider or to a designated clinical coordinator via web interface (27), enabling timely re-engagement.

Response data is stored in client database (23) and may be incorporated into reports generated by reporting system (20). Some versions of system (1) may also include long-term client monitoring capabilities, where productivity optimization system (18) periodically checks in with former clients to assess whether they are maintaining progress or require follow-up care, extending the support provided by system (1) beyond the active treatment phase.

Productivity optimization system (18) may be configured to detect and flag billing mistakes, including both underpayments and overpayments, by cross-referencing billing codes submitted for services rendered against a plurality of reference sources stored in client database (23), including established billing guidelines, historical billing patterns for similar services, insurance claim adjudication data, and service duration records. When a discrepancy is identified between a submitted billing code and one or more of these reference sources, productivity optimization system (18) generates an alert. The alerts may be prioritized based on the potential financial impact of the error, such that errors with a higher potential financial impact are presented to users via web interface (27) with greater prominence or urgency.

For example, a billing code discrepancy that could result in a claim denial for a high-value service episode may be assigned a higher priority than a billing code discrepancy that affects only a minor fee adjustment. The prioritized alerts may be delivered through in-app notifications within web interface (27), email alerts, or SMS messages, depending on user preferences and the urgency level assigned to each alert.

Productivity optimization system (18) may also generate interactive graphs and charts that display the frequency and monetary value of caught billing errors over time, including trend analysis to show improvements in billing accuracy and projected long-term savings, and may produce detailed reports highlighting specific billing mistakes, their causes, and recommended corrective actions.

Productivity optimization system (18) may also feature streamlined reporting capabilities, allowing for the automatic generation of reports on various aspects of the underlying behavioral health practice, such as treatment progress, team activities, and client outcomes. By consolidating key performance indicators into easily digestible formats, productivity optimization system (18) may save time in report preparation and facilitate more effective communication of progress to stakeholders.

### VII. Reporting System

Reporting system (20) of system (1) aggregates and processes information from all other components of system (1) to provide comprehensive reports that offer insights for decision-making and quality improvement in behavioral health settings. In some implementations, reporting system (20) may automatically collect data from client data management system (12), treatment planning system (14), compliance management system (16), and productivity optimization system (18), allowing for the generation of holistic reports encompassing client outcomes, treatment efficacy, compliance metrics, and operational efficiency.

Reporting system (20) may offer a range of report types, including treatment outcome reports that analyze data from client data management system (12) and treatment planning system (14) to evaluate the effectiveness of interventions and track client progress over time, compliance summaries that draw on compliance records (29) and versioned records (31) from compliance management system (16) to highlight adherence to regulatory standards and identify potential areas of risk, and productivity analytics that assess workflow efficiency and resource utilization based on data from productivity optimization system (18).

Reporting system (20) may feature customizable reporting options, allowing healthcare providers to tailor reports to their specific needs, and may incorporate data visualization tools such as interactive charts, graphs, and dashboards that present data in easily digestible formats. By analyzing data from productivity optimization system (18) alongside treatment outcomes, healthcare providers may identify correlations between workflow efficiency and patient progress, leading to targeted improvements in care delivery processes.

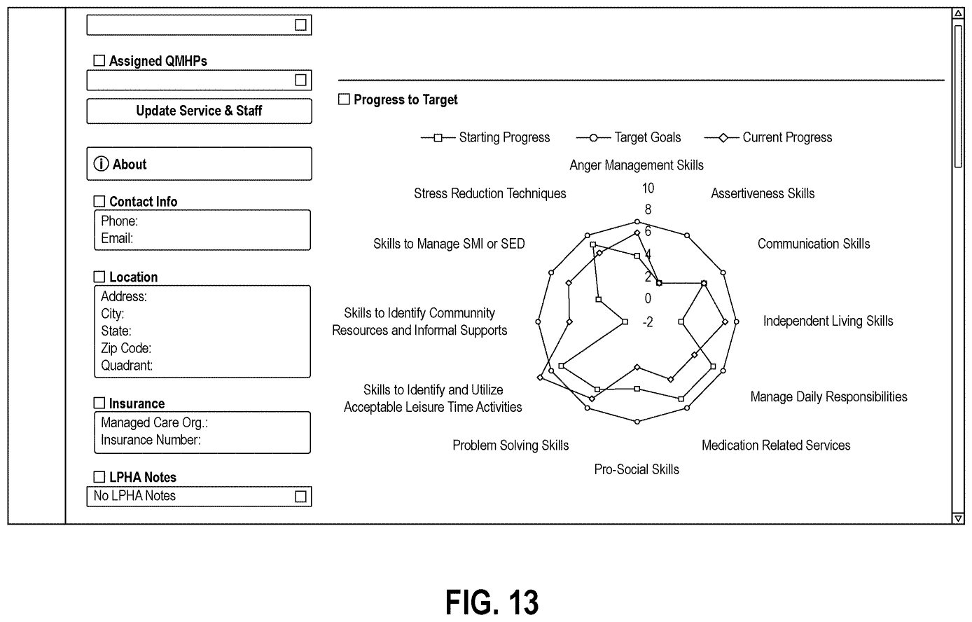

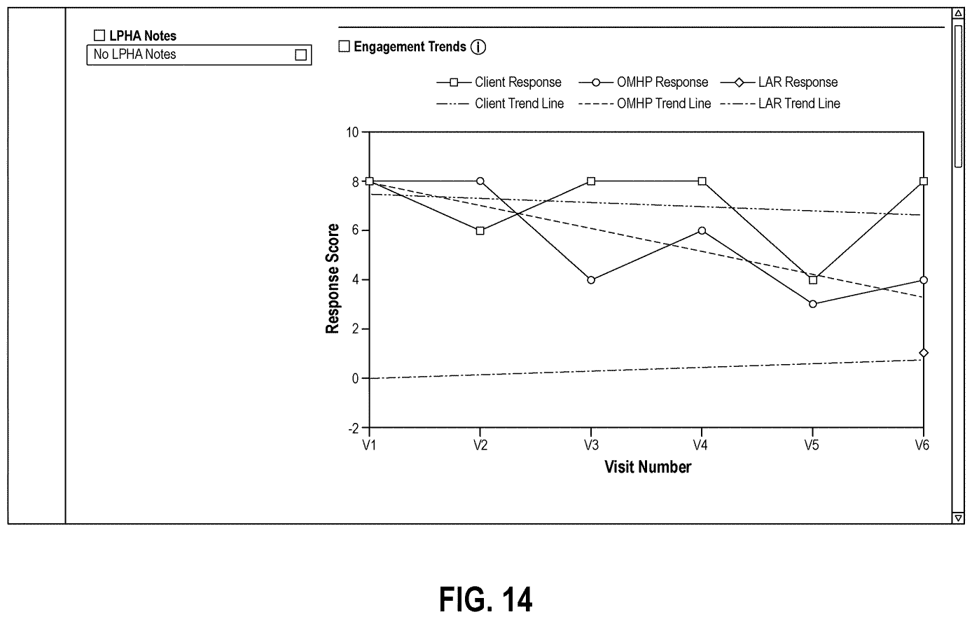

Exemplary web interface (27) screens for reporting system (20) are illustrated in FIGS. 13 and 14. FIG. 13 illustrates a Progress to Target report featuring a multidimensional spider chart (also referred to as a radar chart). The spider chart includes a plurality of axes, where each axis represents a different treatment goal or skill area (e.g., social skills, communication, self-advocacy, goal setting, articulation, emotional regulation). Concentric rings on the spider chart indicate progress levels, ranging from an innermost ring representing an initial baseline to an outermost ring representing a target level. The client's current progress along each axis is plotted on the spider chart, producing a polygon that visually represents the client's overall treatment progress profile. The spider chart enables healthcare providers to quickly identify areas of significant progress as well as areas requiring additional focus, and supports comparison of current status against both the initial baseline and the target goals established in the treatment plan generated by treatment planning system (14). In some cases, the spider chart may overlay multiple polygons, such as a baseline polygon, a current progress polygon, and a target polygon, enabling visual comparison across these three states simultaneously. The spider chart's flexibility permits the customization of displayed metrics based on individual treatment plans or organizational requirements, enhancing its utility across diverse behavioral health scenarios.

FIG. 14 illustrates an Engagement Trends report featuring a detailed line graph based on collected data. This longitudinal visualization tool tracks client engagement over time, incorporating multiple data points such as attendance rates, participation levels, and progress in specific treatment areas. The line graph may include multiple trend lines representing different engagement metrics, allowing for the identification of correlations between various aspects of the client's treatment journey. This type of visualization is particularly valuable for identifying patterns, such as periods of increased or decreased engagement, which can inform adjustments to treatment strategies. The Engagement Trends report may also incorporate interactive elements, allowing users to zoom into specific time periods or toggle between different metrics for more detailed analysis.

### VIII. Additional System(s)/Scalability and Integration

System (1) incorporates scalability and integration features that enable it to adapt and grow alongside organizations of various sizes. As healthcare providers expand their operations, system (1) may accommodate increasing demands by handling larger data volumes, more complex workflows, and a growing user base across various roles and departments without compromising performance or security. System (1) may support the addition of new modules over time, providing flexibility for healthcare providers to customize system (1) to their specific requirements and extend capabilities as their needs evolve. System (1) may also include features to support expansion into new regions, including capabilities for multi-region deployments and localization features that allow healthcare providers to adapt system (1) to different regulatory environments and language requirements as they expand their geographical reach.

In some cases, system (1) may integrate with a wide variety of third-party systems via network (24) or I/O interface (34), allowing for a unified ecosystem of tools and data. This integration capability may enable healthcare organizations to leverage existing software investments and enhance overall functionality by connecting system (1) with electronic health records, billing systems, or specialized behavioral health tools. For example, system (1) may exchange data with an external electronic health record system to import or export client records, or may interface with an external billing system to coordinate billing codes and claim submissions with the billing error detection capabilities of productivity optimization system (18). Other systems (22) may encompass these and other additional functionalities that support the overall operation of system (1), such as integration with external systems or specialized modules for specific behavioral health interventions.

### IX. System and Data Interaction

While any system architecture or system/sub-system organization is contemplated by this disclosure, the embodiment of the present invention shown and described as system (1) comprises several interconnected components that work together to provide comprehensive behavioral health case management: client data management system (12), treatment planning system (14), compliance management system (16), productivity optimization system (18), reporting system (20), and potentially other systems (22). Client data management system (12) includes client database (23), which stores the data objects generated and used by the other components, including treatment plan data records (25), compliance records (29), and versioned records (31).

In operation, client data management system (12) collects and stores client records in client database (23), including demographic information, medical history, and assessment results. Treatment planning system (14) accesses this data and generates a treatment plan data record (25) for each client based at least in part on the set of mental health data stored in client database (23) and a current insurance policy applicable to the client. Compliance management system (16) periodically accesses one or more external sources of insurance policy information (33) via network (24) to obtain an accessed insurance policy, compares the accessed insurance policy against treatment plan data records (25) stored in client database (23) to detect discrepancies, and automatically updates affected treatment plan data records (25) to conform to the accessed insurance policy. When compliance management system (16) performs an update, a compliance record (29) documenting the update is stored in client database (23), and the accessed insurance policy is stored as a versioned record (31), enabling an audit trail linking each treatment plan modification to the specific insurance policy version that prompted it. Compliance management system (16) may also automatically update internal compliance guidelines and rules stored in client database (23), such that future treatment plan data records (25) generated by treatment planning system (14) reflect current insurance requirements without further manual configuration.

Productivity optimization system (18) interacts with client database (23) and treatment planning system (14) to automate scheduling, task allocation, and resource management, performs automated billing error detection by cross-referencing billing codes against reference sources stored in client database (23), and transmits automated post-treatment follow-up communications via network (24) to clients who have exited a treatment program. Reporting system (20) aggregates data from client database (23) and all other components of system (1) to generate comprehensive reports, including treatment outcome reports analyzing treatment plan data records (25), compliance summaries drawing on compliance records (29) and versioned records (31), and productivity analytics incorporating data from productivity optimization system (18). Other systems (22) may encompass additional functionalities that support the overall operation of system (1), such as integration with external systems or specialized modules for specific behavioral health interventions.

The closed-loop compliance process performed by compliance management system (16), in which insurance policy information is periodically retrieved from external sources of insurance policy information (33), compared against treatment plan data records (25), and used to automatically update those records when discrepancies are detected, helps prevent delays in treatment approval and reduces the risk of claim denials. The storage of compliance records (29) and versioned records (31) in client database (23) provides an audit trail that supports regulatory reviews and insurance audits. By ensuring that all components of system (1) have access to up-to-date client information and current insurance requirements stored in client database (23), system (1) supports informed decision-making and personalized, compliant care delivery.

While a specific embodiment of the implementations of system (1) have been described, it will be understood that various modifications may be made without departing from the spirit and scope of the disclosure. Accordingly, other implementations are within the scope of the following claims.

### X. Exemplary Method

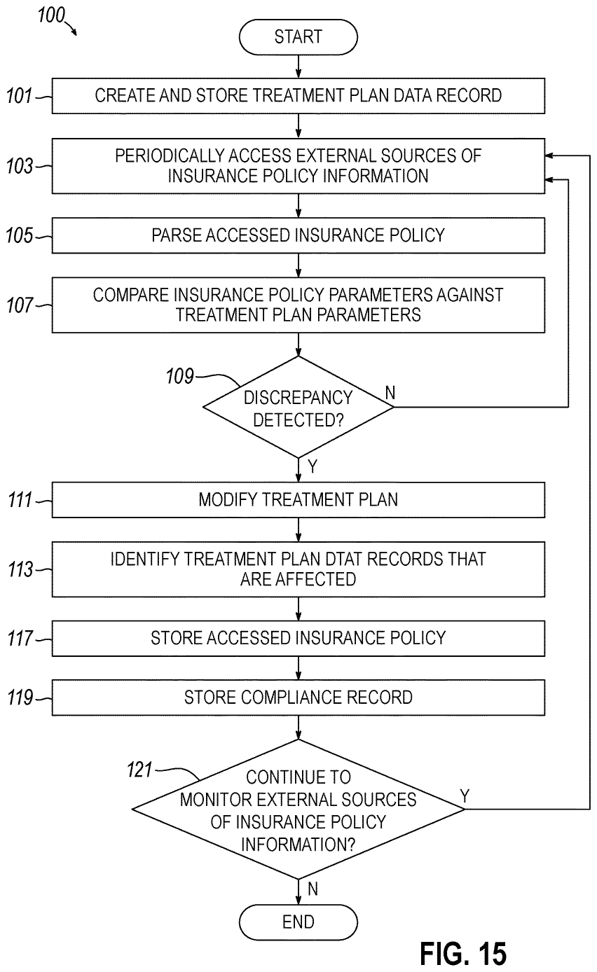

FIG. 15 illustrates an exemplary method (100) for managing mental health data. Method (100) may be performed by system (1) using any combination of client data management system (12), treatment planning system (14), compliance management system (16), and client database (23), operating within operating environment (10) as described herein. While method (100) is described below with reference to specific steps performed in a particular order, it will be understood that some steps may be performed in a different order, performed concurrently, or omitted, depending on the particular implementation.

At step (101), an instance of treatment plan data record (25) comprising a plurality of treatment plan parameters for a client is created and stored in client database (23). Treatment plan data record (25) may be created by treatment planning system (14) based at least in part on the set of mental health data associated with the client stored in client database (23) and on a current insurance policy applicable to the client. The plurality of treatment plan parameters stored in treatment plan data record (25) may include, for example, covered service types, session frequency, session duration limits, required documentation elements, required assessment intervals, billing codes, treatment goals, and assigned providers. In some implementations, treatment plan data record (25) may be generated in part from a customizable treatment template selected by a healthcare provider and automatically populated with data from client database (23). In other implementations, treatment plan data record (25) may be generated from assessment results collected through a behavioral health assessment administered by client data management system (12), such as a CANS, ANSA, or Naomi assessment. Step (101) may be performed once for each new client, or may be repeated when a new treatment plan is created for an existing client (e.g., when a client transitions to a new phase of treatment or when a prior treatment plan expires).

At step (103), compliance management system (16) periodically crawls one or more external sources of insurance policy information (33) via network (24) to obtain an accessed insurance policy. The periodic crawling may occur on a scheduled basis (e.g., daily, weekly, or at any other configurable interval), or may be triggered by an event such as a notification received from an insurance provider, a change detected in a regulatory database, or a manual compliance check initiated by a user via web interface (27). The one or more external sources of insurance policy information (33) may include, for example, insurance provider websites, state regulatory agency websites, federal regulatory databases, APIs provided by insurance providers, or electronic data feeds published by insurance providers or regulatory bodies. In some implementations, compliance management system (16) may crawl multiple external sources during a single periodic cycle to obtain insurance policy information from a plurality of different insurers or regulatory jurisdictions, ensuring broad coverage across the client population served by system (1). In other implementations, compliance management system (16) may query a single API endpoint that aggregates policy information from multiple insurers.

At step (105), the accessed insurance policy obtained at step (103) is parsed to extract a plurality of insurance policy parameters. The insurance policy parameters may include, for example, covered service types, maximum session limits, session duration requirements, required documentation elements, approved billing codes, required assessment protocols, covered treatment modalities, or any other parameters that define the scope and requirements of insurance coverage for behavioral health services. In some implementations, the parsing may involve extracting structured data from a machine-readable format such as an API response, an XML document, or a JSON data feed. In other implementations, the parsing may involve extracting parameters from unstructured or semi-structured data, such as text-based policy documents, regulatory bulletins, or web page content retrieved through crawling. Compliance management system (16) may apply pattern recognition, keyword extraction, or other data processing techniques to identify and extract relevant insurance policy parameters from the accessed insurance policy regardless of the format in which the policy data is received.

At step (107), the plurality of insurance policy parameters extracted at step (105) are compared against the plurality of treatment plan parameters stored in treatment plan data record (25) in client database (23). This comparison may be performed on a parameter-by-parameter basis, where each insurance policy parameter is compared against a corresponding treatment plan parameter to determine whether the treatment plan parameter conforms to the insurance policy parameter. For example, if the accessed insurance policy specifies a maximum of 12 covered sessions per treatment episode, compliance management system (16) may compare this limit against the session frequency parameter stored in treatment plan data record (25) to determine whether the treatment plan exceeds the covered limit. Similarly, if the accessed insurance policy requires a specific type of documentation to be maintained for each session, compliance management system (16) may compare this requirement against the required documentation elements parameter in treatment plan data record (25).

At decision (109), compliance management system (16) determines whether a discrepancy exists between at least one insurance policy parameter and at least one corresponding treatment plan parameter. A discrepancy may exist when a treatment plan parameter exceeds a limit imposed by an insurance policy parameter, when a treatment plan parameter references a service type or billing code that is no longer covered under the accessed insurance policy, when the accessed insurance policy requires a documentation element or assessment protocol that is not reflected in treatment plan data record (25), or when any other misalignment is detected between the treatment plan parameters and the insurance policy parameters. If no discrepancy is detected, method (100) returns to step (103) to continue periodic monitoring of external sources of insurance policy information (33). If a discrepancy is detected, method (100) proceeds to step (111).

At step (111), compliance management system (16) automatically modifies the at least one corresponding treatment plan parameter in treatment plan data record (25) in client database (23) to conform to the at least one insurance policy parameter. The modification may include, for example, reducing a session frequency to comply with a new maximum session limit, updating a billing code to reflect a change in covered service codes, adding a required documentation element that was not previously included in the treatment plan, adjusting a session duration limit to conform to an updated coverage restriction, or any other modification necessary to bring treatment plan data record (25) into conformity with the accessed insurance policy. In some implementations, compliance management system (16) performs the modification automatically without user intervention. In other implementations, compliance management system (16) may present the proposed modification to a healthcare provider via web interface (27) for review and approval before the modification is finalized in client database (23).

At step (113), compliance management system (16) identifies additional treatment plan data records (25) stored in client database (23) that are affected by the at least one insurance policy parameter that triggered the discrepancy detected at decision (109). For example, if the accessed insurance policy reflects a change in coverage requirements that applies to all clients enrolled under a particular insurance plan, compliance management system (16) may query client database (23) to identify all treatment plan data records (25) associated with clients covered by that insurance plan. Similarly, if a state regulatory body has updated a regulation that applies to all clients receiving a particular type of behavioral health service within that state, compliance management system (16) may identify all treatment plan data records (25) that include that service type for clients within the affected jurisdiction.

At step (115), compliance management system (16) automatically modifies each affected treatment plan data record (25) identified at step (113) to conform to the at least one insurance policy parameter, in a manner consistent with the modification performed at step (111). This batch modification capability enables compliance management system (16) to propagate a single detected insurance policy change across all affected treatment plans stored in client database (23) without requiring a healthcare provider to manually review and update each treatment plan individually.

At step (117), the accessed insurance policy obtained at step (103) is stored in client database (23) as a versioned record (31). Versioned record (31) includes the content of the accessed insurance policy, an identifier of the external source of insurance policy information (33) from which it was retrieved, and a retrieval timestamp. By storing each accessed insurance policy as a distinct versioned record (31), client database (23) accumulates a chronological history of insurance policies over time, enabling system (1) to maintain a record of the insurance policy landscape as it existed at any given point in time.

At step (119), a compliance record (29) is stored in client database (23) for each treatment plan data record (25) modified at steps (111) and (115). Each compliance record (29) associates the modified treatment plan data record (25) with the versioned record (31) stored at step (117) and includes a timestamp indicating when the modification was performed. Each compliance record (29) may further include the detected discrepancy, the original treatment plan parameters prior to the modification, and the updated treatment plan parameters following the modification. The association between compliance records (29) and versioned records (31) enables system (1) to trace any treatment plan modification back to the specific version of the insurance policy that required it, supporting insurance audits, regulatory reviews, and compliance investigations.

At decision (121), compliance management system (16) determines whether to continue periodic monitoring of external sources of insurance policy information (33). In most implementations, method (100) proceeds back to step (103) to continue the periodic monitoring cycle, as insurance requirements may change at any time and ongoing monitoring helps ensure that treatment plan data records (25) remain in conformity with current insurance policies. If periodic monitoring is to be discontinued (e.g., because system (1) is being taken offline for maintenance, because a particular insurance plan has been terminated, or because a healthcare provider has opted out of automated compliance monitoring), method (100) ends at decision (121).

### XI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system for the management of mental health data, comprising: (a) a client data management system comprising a client database configured to store a set of mental health data associated with a client; (b) a treatment planning system operatively coupled to the client data management system, wherein the treatment planning system is configured to generate a treatment plan data record based at least in part on the set of mental health data associated with the client and a current insurance policy; and (c) a compliance management system operatively coupled to the treatment planning system and configured to: (i) periodically access, via a network, an external source of insurance policy information to obtain an accessed insurance policy, (ii) compare the treatment plan data record with the accessed insurance policy to detect a discrepancy, and (iii) in response to detecting the discrepancy, automatically update the treatment plan data record in the client database to conform to the accessed insurance policy.

### Example 2

The system of any of the previous or subsequent Examples, wherein the compliance management system is further configured to store a compliance record in the client database documenting the update to the treatment plan data record.

### Example 3

The system of any of the previous or subsequent Examples, wherein the compliance record includes the discrepancy and a timestamp.

### Example 4

The system of any of the previous or subsequent Examples, wherein automatically updating the treatment plan data record comprises modifying at least one of a covered service type, a session frequency, a session duration limit, a required documentation element, or a billing code stored in the treatment plan data record.

### Example 5

The system of any of the previous or subsequent Examples, wherein periodically accessing the external source of insurance policy information comprises crawling, via the network, a plurality of external sources.

### Example 6

The system of any of the previous or subsequent Examples, wherein the plurality of external sources includes at least one insurance provider website and at least one state regulatory agency website.

### Example 7

The system of any of the previous or subsequent Examples, wherein the compliance management system is further configured to extract a set of insurance policy parameters from the plurality of external sources, and wherein comparing the treatment plan data record with the accessed insurance policy comprises comparing the set of insurance policy parameters against a set of corresponding parameters stored in the treatment plan data record.

### Example 8

The system of any of the previous or subsequent Examples, wherein periodically accessing the external source of insurance policy information comprises querying an application programming interface (API) provided by an insurance provider to retrieve a set of current insurance policy parameters associated with the client.

### Example 9

The system of any of the previous or subsequent Examples, wherein periodically accessing the external source of insurance policy information comprises receiving an electronic data feed from an insurance provider via the network, and wherein the compliance management system is configured to process incoming data from the electronic data feed without user initiation.

### Example 10

The system of any of the previous or subsequent Examples, wherein periodically accessing the external source of insurance policy information comprises receiving an electronic data feed from a regulatory body via the network, and wherein the compliance management system is configured to process incoming data from the electronic data feed without user initiation.

### Example 11

The system of any of the previous or subsequent Examples, wherein the compliance management system is further configured to, in response to detecting the discrepancy, identify a plurality of treatment plan data records stored in the client database that are affected by the accessed insurance policy, and automatically update each of the plurality of treatment plan data records to conform to the accessed insurance policy.

### Example 12

The system of any of the previous or subsequent Examples, wherein the compliance management system is further configured to store the accessed insurance policy in the client database in a versioned record, wherein each update to a treatment plan data record is associated with a corresponding versioned record of the accessed insurance policy that prompted the update.

### Example 13

The system of any of the previous or subsequent Examples, wherein the treatment planning system further comprises a web interface configured to embed hyperlinks to an online section of a governing administrative code applicable to the treatment plan data record, wherein the hyperlinks are dynamically generated based on a jurisdiction, a service type, and content of the treatment plan data record.

Example 14

The system of any of the previous or subsequent Examples, wherein the client data management system is further configured to guide a user through a behavioral health assessment by presenting, for each assessment question in a plurality of assessment questions, clarifying question information, simplified language, and a set of color-coded individual selection buttons indicating severity, and wherein a set of results of the behavioral health assessment are stored in the client database, wherein the set of results are used by the treatment planning system to generate the treatment plan data record.

### Example 15

The system of any of the previous or subsequent Examples, wherein the behavioral health assessment comprises at least one of a Child and Adolescent Needs and Strengths (CANS) assessment, an Adult Needs and Strengths Assessment (ANSA), or a Naomi Assessment, and wherein the client data management system records both a numeric value and a timestamp in the client database for each color-coded selection button selected by the user.

### Example 16

The system of any of the previous or subsequent Examples, wherein the system is further configured to maintain an audit log of user actions, data modifications, and compliance-related decisions in the client database, wherein the system is further configured to provide a specialized interface for an authorized auditor to access the audit log stored in the client database.

### Example 17

The system of any of the previous or subsequent Examples, further comprising a productivity optimization system configured to perform an automated billing error detection action by cross-referencing a set of billing codes against a plurality of reference sources stored in the client database to generate a detected billing error.

### Example 18

The system of any of the previous or subsequent Examples, wherein the productivity optimization system is configured to generate a potential financial impact of a detected billing error, wherein the productivity optimization system is configured to generate an alert associated with the detected billing error, wherein the alert is prioritized based on the potential financial impact of the detected billing error.

### Example 19

A computer-implemented method for managing mental health data, comprising: (a) storing, in a client database residing on a mass storage memory device, a treatment plan data record comprising a plurality of treatment plan parameters for a client; (b) periodically crawling, by a compliance management system via a network, one or more external sources of insurance policy information to obtain an accessed insurance policy; (c) parsing the accessed insurance policy to extract a plurality of insurance policy parameters; (d) comparing the plurality of insurance policy parameters against the plurality of treatment plan parameters stored in the treatment plan data record to detect a discrepancy between at least one insurance policy parameter and at least one corresponding treatment plan parameter; (e) in response to detecting the discrepancy, automatically modifying the at least one corresponding treatment plan parameter in the treatment plan data record stored in the client database to conform to the at least one insurance policy parameter; and (f) storing a compliance record in the client database, the compliance record associating the modified treatment plan data record with a versioned record of the accessed insurance policy and including a timestamp.

### Example 20

The method or system of any of the previous Examples, further comprising, in response to detecting the discrepancy, identifying a plurality of treatment plan data records stored in the client database that are affected by the at least one insurance policy parameter, and automatically modifying each of the plurality of treatment plan data records to conform to the at least one insurance policy parameter, and storing a respective compliance record for each modified treatment plan data record.

### XII. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A system for the management of mental health data, comprising:
(a) a client data management system comprising a client database configured to store a set of mental health data associated with a client;
(b) a treatment planning system operatively coupled to the client data management system, wherein the treatment planning system is configured to generate a treatment plan data record based at least in part on the set of mental health data associated with the client and a current insurance policy; and
(c) a compliance management system operatively coupled to the treatment planning system and configured to:
(i) periodically access, via a network, an external source of insurance policy information to obtain an accessed insurance policy,
(ii) compare the treatment plan data record with the accessed insurance policy to detect a discrepancy, and
(iii) in response to detecting the discrepancy, automatically update the treatment plan data record in the client database to conform to the accessed insurance policy.

2. The system of claim 1, wherein the compliance management system is further configured to store a compliance record in the client database documenting the update to the treatment plan data record.

3. The system of claim 2, wherein the compliance record includes the discrepancy and a timestamp.

4. The system of claim 1, wherein automatically updating the treatment plan data record comprises modifying at least one of a covered service type, a session frequency, a session duration limit, a required documentation element, or a billing code stored in the treatment plan data record.

5. The system of claim 1, wherein periodically accessing the external source of insurance policy information comprises crawling, via the network, a plurality of external sources.

6. The system of claim 5, wherein the plurality of external sources includes at least one insurance provider website and at least one state regulatory agency website.

7. The system of claim 5, wherein the compliance management system is further configured to extract a set of insurance policy parameters from the plurality of external sources, and wherein comparing the treatment plan data record with the accessed insurance policy comprises comparing the set of insurance policy parameters against a set of corresponding parameters stored in the treatment plan data record.

8. The system of claim 1, wherein periodically accessing the external source of insurance policy information comprises querying an application programming interface (API) provided by an insurance provider to retrieve a set of current insurance policy parameters associated with the client.

9. The system of claim 1, wherein periodically accessing the external source of insurance policy information comprises receiving an electronic data feed from an insurance provider via the network, and wherein the compliance management system is configured to process incoming data from the electronic data feed without user initiation.

10. The system of claim 1, wherein periodically accessing the external source of insurance policy information comprises receiving an electronic data feed from a regulatory body via the network, and wherein the compliance management system is configured to process incoming data from the electronic data feed without user initiation.

11. The system of claim 1, wherein the compliance management system is further configured to, in response to detecting the discrepancy, identify a plurality of treatment plan data records stored in the client database that are affected by the accessed insurance policy, and automatically update each of the plurality of treatment plan data records to conform to the accessed insurance policy.

12. The system of claim 1, wherein the compliance management system is further configured to store the accessed insurance policy in the client database in a versioned record, wherein each update to a treatment plan data record is associated with a corresponding versioned record of the accessed insurance policy that prompted the update.

13. The system of claim 1, wherein the treatment planning system further comprises a web interface configured to embed hyperlinks to an online section of a governing administrative code applicable to the treatment plan data record, wherein the hyperlinks are dynamically generated based on a jurisdiction, a service type, and content of the treatment plan data record.

14. The system of claim 1, wherein the client data management system is further configured to guide a user through a behavioral health assessment by presenting, for each assessment question in a plurality of assessment questions, clarifying question information, simplified language, and a set of color-coded individual selection buttons indicating severity, and wherein a set of results of the behavioral health assessment are stored in the client database, wherein the set of results are used by the treatment planning system to generate the treatment plan data record.

15. The system of claim 14, wherein the behavioral health assessment comprises at least one of a Child and Adolescent Needs and Strengths (CANS) assessment, an Adult Needs and Strengths Assessment (ANSA), or a Naomi Assessment, and wherein the client data management system records both a numeric value and a timestamp in the client database for each color-coded selection button selected by the user.

16. The system of claim 1, wherein the system is further configured to maintain an audit log of user actions, data modifications, and compliance-related decisions in the client database, wherein the system is further configured to provide a specialized interface for an authorized auditor to access the audit log stored in the client database.

17. The system of claim 1, further comprising a productivity optimization system configured to perform an automated billing error detection action by cross-referencing a set of billing codes against a plurality of reference sources stored in the client database to generate a detected billing error.

18. The system of claim 17, wherein the productivity optimization system is configured to generate a potential financial impact of a detected billing error, wherein the productivity optimization system is configured to generate an alert associated with the detected billing error, wherein the alert is prioritized based on the potential financial impact of the detected billing error.

19. A computer-implemented method for managing mental health data, comprising:
(a) storing, in a client database residing on a mass storage memory device, a treatment plan data record comprising a plurality of treatment plan parameters for a client;
(b) periodically crawling, by a compliance management system via a network, one or more external sources of insurance policy information to obtain an accessed insurance policy;
(c) parsing the accessed insurance policy to extract a plurality of insurance policy parameters;
(d) comparing the plurality of insurance policy parameters against the plurality of treatment plan parameters stored in the treatment plan data record to detect a discrepancy between at least one insurance policy parameter and at least one corresponding treatment plan parameter;
(e) in response to detecting the discrepancy, automatically modifying the at least one corresponding treatment plan parameter in the treatment plan data record stored in the client database to conform to the at least one insurance policy parameter; and
(f) storing a compliance record in the client database, the compliance record associating the modified treatment plan data record with a versioned record of the accessed insurance policy and including a timestamp.

20. The computer-implemented method of claim 19, further comprising, in response to detecting the discrepancy, identifying a plurality of treatment plan data records stored in the client database that are affected by the at least one insurance policy parameter, and automatically modifying each of the plurality of treatment plan data records to conform to the at least one insurance policy parameter, and storing a respective compliance record for each modified treatment plan data record.
